(19) United States
(12) Patent Application Publication
(10) Pub. No.: US 2010/0191068 A1
(43) Pub. Date: Jul. 29, 2010

(54) SPACER BLOCK WITH SENSOR FOR TOTAL KNEE ARTHROPLASTY

(75) Inventors: Mark Alan Bewernitz, Gainesville, FL (US); Peter F. Gearen, Gainesville, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Florida Research Foundation, Inc.

(21) Appl. No.: 11/697,081

(22) Filed: Apr. 5, 2007

Related U.S. Application Data
(60) Provisional application No. 60/790,139, filed on Apr. 7, 2006.

(51) Int. Cl. A61B 5/00; A61B 17/58

(52) U.S. Cl. 600/300; 606/88

(57) ABSTRACT

Embodiments of the present invention relate to a force sensing system and method for total knee arthroplasty. For example, a force sensing system includes a spacer block that is adapted to be positioned adjacent a resected distal femur and a resected proximal tibia. The spacer block is configured to receive a first force applied by the resected distal femur and a second force applied by the resected proximal tibia. The force sensing system also includes at least one sensor that is associated with the spacer block and that is adapted to measure one or more forces exerted on the spacer block. Moreover, the force sensing system includes a display that is adapted for displaying an indication of the one or more forces measured by the at least one sensor.

### CROSS REFERENCE TO RELATED APPLICATION

The present application claims priority from U.S. Provisional Application No. 60/790,139 filed Apr. 7, 2006, the contents of which are incorporated herein by reference.

### BACKGROUND

During total knee arthroplasty, portions of the distal femur and proximal tibia are excised to be replaced by the knee implant. The dimensions of the excised regions should typically match the dimensions of the knee joint replacement so as to not alter the stress on the collateral ligaments that hold the joint together. If not enough bone material is excised then an “overstuffing” situation occurs which stretches the collateral ligaments leading to post-operative pain, stiffness, and reduced knee range of motion for the patient. If too much bone material is excised, then “under-stuffing” will occur leading to looseness of the collateral ligaments resulting in lack of support in the knee joint and possibly future risk of injury. Another source of post-operative pain and reduced range of motion is the accidental creation of “up-sloping” or “down-sloping” on the femoral or tibial resection plane. During the resection of bone, care is taken to generate resection planes that are normal to the centerline of each bone. Slight sloping of the resection plane can lead to reduced flexion or extension space and therefore limited flexion or extension after surgery.

Traditionally, these conditions are avoided by using angle-measuring devices (to generate non-sloped resection planes) and spacer blocks (to generate exacting resection dimensions for the insertion of the replacement joint). However, due to differences in joint structure between the individuals and large amount of “play” or wiggle room the joint has when surgeons are artificially flexing and extending the joints to test during the procedure, there are still occurrences of the above-described negative conditions. The surgeons flex and extend the leg to get a feel for how tightly the replacement joint fits but ultimately rely on instincts, rather than on any measurable data to determine if there will be post-operative ligament tension problems.

Therefore, there is a need for systems and methods for assisting a surgeon during total knee arthroplasty to reduce the incidence of negative post-operative effects.

### BRIEF SUMMARY

Embodiments of the present invention relate to force sensing systems and methods for assisting a surgeon during total knee arthroplasty. According to one embodiment, a force sensing system includes a spacer block that is adapted to be positioned adjacent to a resected distal femur and a resected proximal tibia. The spacer block is configured to receive one or more forces applied by the resected distal femur and/or the resected proximal tibia. The force sensing system also includes a sensor array that is associated with said spacer block and that is adapted to measure one or more forces exerted on the spacer block. In addition, the force sensing system includes a processing element configured to receive information from the sensor array and generate information indicative of the one or more forces exerted on the spacer block.

According to aspects of the force sensing system, the system may further include a display configured for displaying the information indicative of the one or more forces exerted on the spacer block. In addition, the display may be configured to display a two-dimensional image indicative of a contact pressure on the spacer block. Furthermore, the display may be configured to display an indication of an anisotropic contact pressure on the spacer block. According to an additional aspect, the sensor array may be integrated with the spacer block.

### BRIEF DESCRIPTION OF THE DRAWINGS

Having thus described the invention in general terms, reference will now be made to the accompanying drawings, which are not necessarily drawn to scale, and wherein:

FIG. 1 depicts a system for assisting a surgeon during total knee arthroplasty according to a particular embodiment of the invention.

FIG. 2 depicts a perspective view of a spacer block according to one embodiment of the invention.

FIG. 3 depicts a side view of the spacer block shown in FIG. 2.

FIGS. 4a-4d illustrate a method for using a spacer block for total knee arthroplasty according to a particular embodiment of the invention.

### DETAILED DESCRIPTION OF VARIOUS EMBODIMENTS OF THE INVENTION

The present invention will now be described more fully hereinafter with reference to the accompanying drawings, in which various embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

Various embodiments of the present invention are configured to assist a surgeon during total knee arthroplasty (“TKA”). In particular, certain embodiments of the present invention address the problem associated with post-TKA collateral ligament tightness or looseness, and/or reduced flexion or extension of the knee joint. According to one embodiment of the present invention, a spacer block is provided that includes one or more sensors, and that is adapted to enable a surgeon to more accurately assess the resection planes of the joint to accommodate the dimensions of the replacement implant while monitoring the stress on the collateral ligaments (e.g., via the spacer block's one or more sensors). As such, various embodiments serve to reduce the incidence of the painful and dangerous post-operative effects due to improper fitting of the replacement implant.

### More Detailed Description

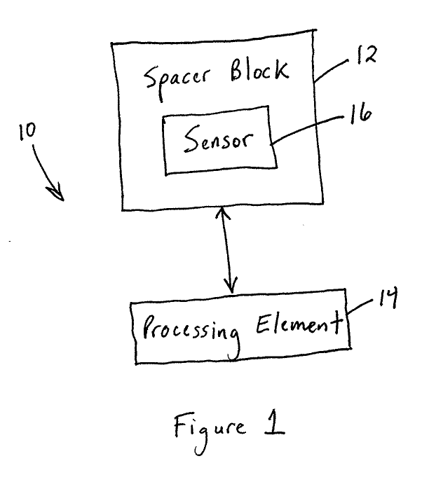

As noted above, various embodiments of the present invention provide techniques for assisting a surgeon during TKA. Referring to the figures and, in particular, FIG. 1, there is shown a system 10 for assisting a surgeon during TKA, where the system generally includes a spacer block 12 and a processing element 14 that are in communication with one another. The spacer block 12 includes at least one sensor 16 that is capable of acquiring data indicative of a force exerted on the spacer block by the resected distal femur and/or the resected proximal tibia. The processing element 14 is capable of then generating data indicative of a contact pressure on the spacer block 12 based on the data acquired by the at least one sensor 16.

Spacer Block

A spacer block 12 according to particular embodiments is adapted to be used during TKA to measure the spacing between the resection planes of the distal femur and the proximal tibia. Thus, each of the distal femur and proximal tibia are resected in order to provide proper spacing and alignment for a replacement implant to be positioned therebetween. Furthermore, in particular embodiments, the spacer block 12 is adapted to ensure that there is not only adequate space for the replacement implant, but also that the resected planes are substantially parallel and accommodate a possible posterior slope.

During use, the spacer block 12 is typically positioned between the resected femur and tibia when the leg is fully extended. As such, when in this configuration, the spacer block 12 is typically adjacent to, and in direct contact with, the resected planes of each of the femur and tibia. The spacer block 12 is positioned adjacent to the resected femur and tibia for determining the spacing between the resected femur and tibia and the contact force and/or pressure on the spacer block. The spacer block 12 is preferably adapted to be easily removed (e.g., via a sliding movement) from between the resected femur and tibia in order to make subsequent cuts or adjustments to the replacement implant.

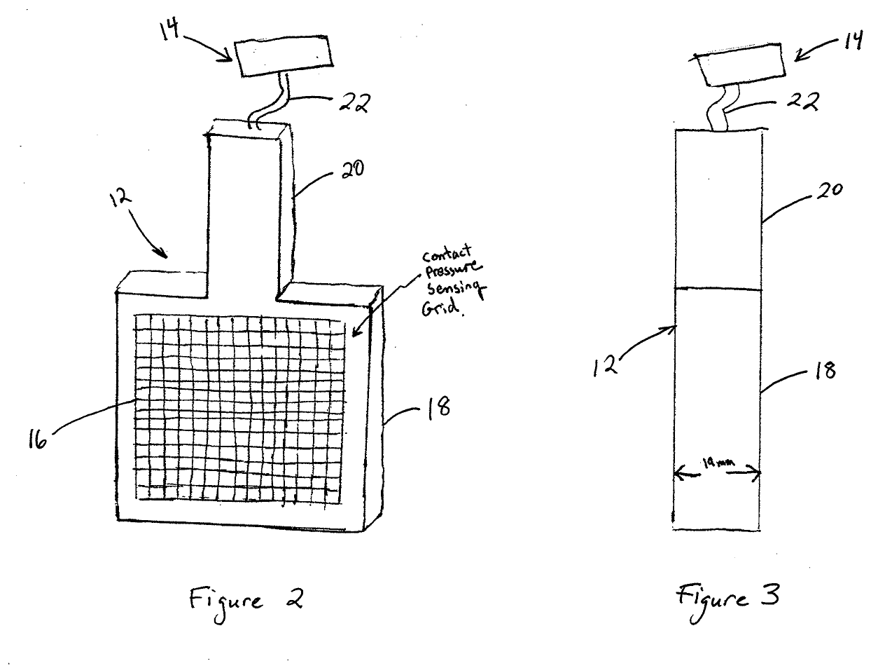

According to one embodiment of the present invention shown in FIGS. 2 and 3, the spacer block 12 includes a sensing portion 18 and a gripping portion 20. The sensing portion 18 shown in FIG. 2 includes an array of sensors 16 which may, for example, be arranged in a grid or other suitable geometrical arrangement. In this embodiment, the sensing portion 18 is generally rectangular (e.g., square) in cross section and includes a first (e.g., substantially planar) surface that is adapted to engage a resected femur and a second (e.g., substantially planar) surface that is adapted to engage a resected tibia. The first and second surfaces are preferably on opposite sides of the spacer block 12. The gripping portion 20 extends outwardly from the sensing portion 18 and allows the surgeon to manipulate the spacer block 12. In the embodiment shown in FIG. 2, a communication cable 22 extends from the gripping portion 22 to a processing element 14 such that data acquired by the sensor 16 can be communicated to the processing element.

According to one embodiment of the present invention, the spacer block 12 is less than 25 mm in thickness (and preferably about 19 mm in thickness), although any suitable thickness may be utilized depending on the type of replacement implant being used. Similarly, although various dimensions of spacer blocks 12 may be employed, the spacer block may be about 5-8 cm in length and width according to one particular embodiment (e.g., 7 cm in width and 6.25 cm in length). Therefore, the spacer block 12 may be at least as large as the leading edges of the femoral and tibial planes. For instance, the spacer block 12 may be about 2-5 cm in length and width in order to substantially conform to the resected leading edges of the femur and tibia. In addition, the spacer block 12 is typically a polymeric or metallic material that is capable of withstanding the forces applied by the femur and tibia thereon, while also being biocompatible and resistant to corrosion.

The spacer block 12 shown in FIGS. 2 and 3 is not meant to be limiting as the spacer block may be various sizes and configurations in additional aspects of the present invention. For instance, the sensing portion 18 may include one or more sensors 16, and the gripping portion 20 may be unnecessary where the surgeon is able to grip the sides of the spacer block 12 in order to manipulate the spacer block. Furthermore, the sensing portion 18 is not limited to the illustrated configuration, as the sensing portion may be various cross sections depending on the patient, type of sensor, desired precision, and/or other factors specific to a particular surgeon. For example, the spacer block 12 could be oval or egg-shaped to conform to the shape of the resected bone. In addition, embodiments of the present invention may involve modifying one or more off-the-shelf spacer blocks 12 to include one or more sensors 16 (e.g., pressure sensors). In this regard, the spacer block 12 can be modified to include one or more sensors 16 such as by directly attaching the sensor to the spacer block.

Sensor

In particular embodiments, the sensors 16 on the sensor portion 18 can be any suitable sensor for acquiring or otherwise outputting data indicative of the forces applied by the resected distal femur and/or the resected proximal tibia on the spacer block 12. For example, one or more of the sensors 16 may be a pressure sensor. In various embodiments, the various sensors 16 may be carried by, embedded within, attached adjacent (e.g., to), integral with, or otherwise associated with the spacer block 12. Also, the sensors 16 may be permanently or temporarily associated with the spacer block 12 and may be positioned on all or a portion of the spacer block. Moreover, the sensors 16 may be located at various positions in relation to the spacer block 12. For example, a particular sensor 16 may be located approximately in the center of the spacer block 12 and/or in respective corners of the spacer block. In particular embodiments, one or more sensors (e.g., pressure sensors) are disposed on: (1) a first (e.g., substantially planar) surface that is adapted to engage a patient's resected femur and/or (2) a second (e.g., substantially planar) surface that is adapted to engage a resected tibia. Alternatively, the sensor may be embedded within the spacer block 12.

The spacer block 12 preferably includes a single sensor 16 but may include an array of sensors, as shown in FIG. 2. In particular embodiments, each sensor 16 of the array of sensors can acquire data indicative of the forces applied by the resected femur and tibia such that a two-dimensional image can be generated that is indicative of the contact pressure at a plurality of points on the surface of the spacer block 12. The contact pressure may, for example, be determined by calculating the contact pressure associated with the force on each individual sensor 16 or by calculating the force per unit area over a specified area on the spacer block 12 corresponding to one or more sensors. As such, in particular embodiments, not only can up-sloping or down-sloping be detected, but also any other imbalances indicated on the image. The surgeon may then take the necessary steps to reduce the pressure imbalances on the spacer block 12 such as by making further cuts on the femur or tibia or adjusting the spacing on the replacement implant.

The sensor 16 can be, for example, any suitable sensor capable of detecting and acquiring data indicative of forces applied by each of the resected femur and tibia on the spacer block 12. Suitable sensors include, for example, a piezoelectric sensor, a strain gauge, a transducer, a load cell, or the like. Moreover, embodiments of the present invention also contemplate that the sensor 16 can directly acquire data indicative of pressure, such as where a sensor is sized and configured to conform to all or a portion of the spacer block 12 such that the force per unit area applied by the resected femur and tibia onto the spacer block can be determined. For example, each sensor 16 of an array or a sensor associated with one or more quadrants of the spacer block 12 may acquire data indicative of the contact pressure on the spacer block.

Processing Element

As described above, one embodiment provides a system 10 for carrying out methods of the present invention. FIG. 1 illustrates at least one sensor 16 (e.g., an array of sensors) that is in communication with a processing element 14. The sensor 16 may be, for example, directly connected to the processing element 14 (see FIGS. 2 and 3) or may be adapted to remotely communicate therewith, such as via wireless or network communications. The sensor 16 may also include internal processing capabilities. The sensor 16 preferably communicates data associated with the patient to the processing element 14 in real time, although batch processing may be implemented if desired.

The processing element 14 is typically a computer, such as a personal computer or workstation, although the processing element may be any device capable of performing embodied methods of the present invention. For instance, the processing element 14 may be a portable device, such as a laptop computer, a personal data assistant, or a readout device capable of displaying data indicative of the contact force and/or pressure between the resected femur and tibia and the spacer block 12. In various embodiments, the processing element 14 is adapted to receive data from one or more sensors 16 on the spacer block 12, to process this data, and to convey this data to a user (e.g., via a graphical display or printout). Embodiments of the present invention also contemplate that the sensor 16 utilized may be capable of processing an output indicative of the contact force and/or pressure between the resected femur and tibia and the spacer block 12, such that the sensor includes an internal processing element. In such embodiments, a readout device may be used to display the data processed by the processing element associated with each sensor 16.

The processing element 14 may include any number of conventional hardware and software components. For example, the processing element 14 could include memory (e.g., RAM), mass storage (e.g., magnetic hard disk or optical storage disk), I/O controller, network interface (e.g., Internet, intranet, or extranet), bus for transferring data or power between processing element components or between processing elements, and/or graphical interface. The graphical interface, as known to those of skill in the art, may provide methods for displaying images generated by the processing element 14 onto a monitor or similar viewing device, as well as interacting with the images. In particular embodiments, the processing element 14 can generate and display various types of images, such as a two-dimensional array image or other images that facilitate the analysis of the acquired data. In addition, the processing element 14 includes a processor that may be adapted to execute one or more applications (e.g., programs) and a standard operating system. For instance, the processing element 14 may employ various software programs for processing and displaying the data acquired by the sensors 16.

As mentioned above, in particular embodiments, the system may be configured to display images representing data from the system's various sensors in real time such that a real-time video display of the captured data may be shown. Also, in particular embodiments, the system is configured to allow a user to capture one or more still images of the data and, for example, to display the still images on a display screen or print the images. However, it should also be understood that the system may be adapted to collect data from the system's sensors at pre-determined times (e.g., while the patient's leg is fully extended) and then to send the collected data to the processing element for display by the graphical interface or for output by an output device, such as a printer. Therefore, although a graphical interface may be preferred in some applications, it is possible to incorporate the processing element 14 without a display and to instead provide a printout of the image(s) or data, or to utilize any other technique for viewing images of the acquired data. In particular embodiments, the system may be adapted to maintain a permanent record of the acquired data for future use or record keeping (e.g., by storing the acquired data in memory), which may allow users to later access previously generated data and/or images.

Furthermore, in particular embodiments, the processing element 14 may be capable of generating and displaying one-, two-, and three-dimensional images. According to one embodiment of the present invention, the processing element 14 is capable of generating a two-dimensional image using an array of sensors 16, such as that shown in FIG. 2. The two-dimensional image may, for example, illustrate anisotropic contact pressure on the spacer block 12 that is indicative of up-sloping or down-sloping resection planes. The two-dimensional image may be displayed in any of various formats, such as by incorporating shading or a color-coded distribution of the individual contact forces and/or pressure associated with each sensor. Additionally, the processing element 14 may generate an average contact force and/or pressure over the spacer block or perform statistical analysis of the data acquired by the sensors 16. The processing element 14 may also be configured for generating various three-dimensional images, such as a three-dimensional contact pressure distribution over the spacer block 12.

Exemplary Method

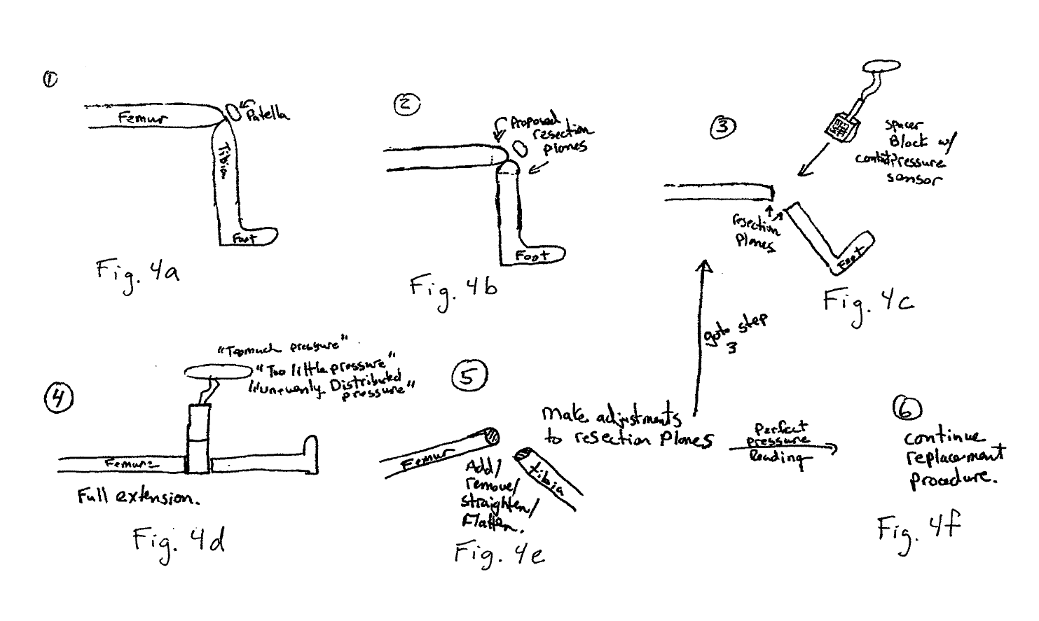

FIGS. 4a-4d illustrate an exemplary method for implementing the system 10 to assist a surgeon during TKA according to one embodiment of the present invention. Namely, the method includes positioning a patient's leg in a generally flexed position and resecting the patient's distal femur and proximal tibia along respective resection planes. The spacer block 12 is then positioned adjacent to (e.g., between) the resection planes of the distal femur and proximal tibia so that at least one pressure sensor disposed on a surface of the spacer block measures the pressure at at least one point between the distal femur and proximal tibia (and preferably transmit this data to a processor, which displays data from the pressure sensor(s) on an appropriate display screen. The patient's leg is then moved to an extended (e.g., fully extended) position.

Once the patient's leg is in an extended (e.g., fully extended) position, the surgeon performing the procedure then views data from the sensors on the system's display screen. If the data on the system's display screen indicates that the contact pressure between the distal femur and proximal tibia is too high or low, or if the slope is inappropriate, the surgeon may then make adjustments to the resection planes and/or the replacement implant until a desired contact pressure is obtained. For instance, the surgeon may make further resection cuts to the femur and tibia if the contact pressure is too high and, similarly, the surgeon may add layers to the femoral and/or tibial implant components if the contact pressure is too low. When a desired contact pressure is indicated, the surgeon can continue with the TKA procedure. This may serve to reduce the incidence of post-operative complications associated with the procedure.

### Conclusion

Many modifications and other embodiments of the invention will come to mind to one skilled in the art to which this invention pertains having the benefit of the teachings presented in the foregoing descriptions and the associated drawings. For example, as will be understood by one skilled in the relevant field in light of this disclosure, the invention may take form in a variety of different mechanical and operational configurations. Therefore, it is to be understood that the invention is not to be limited to the specific embodiments disclosed and that modifications and other embodiments are intended to be included within the scope of the appended exemplary concepts. Although specific terms are employed herein, they are used in a generic and descriptive sense only and not for the purposes of limitation.

1. A force sensing system comprising:
a spacer block that is adapted to be positioned adjacent a resected distal femur and a resected proximal tibia, said spacer block being configured to receive a first force applied by said resected distal femur and a second force applied by said resected proximal tibia;
at least one sensor that is associated with said spacer block and that is adapted to measure one or more forces exerted on said spacer block; and
a display that is adapted for displaying an indication of said one or more forces measured by said at least one sensor.

2. The force sensing system of claim 1, wherein said force sensing system further comprises a processing element that is adapted for:
receiving information, from said sensor, regarding said one or more forces; and
generating a graphical display of said information on said display.

3. The system according to claim 2, wherein said graphical display comprises a two-dimensional image indicative of a contact pressure on said spacer block.

4. The system according to claim 2, wherein said graphical display comprises an indication of an anisotropic contact pressure on said spacer block.

5. The system according to claim 1, wherein said at least one sensor is integrated with said spacer block.

6. The system according to claim 1, wherein said at least one sensor comprises an array of sensors arranged in a grid on said spacer block.

7. A method for assisting a surgeon during total knee arthroplasty comprising:
positioning a spacer block adjacent a resected distal femur and a resected proximal tibia, the spacer block configured to receive at least one force applied by each of the resected distal femur and the resected proximal tibia;
using at least one sensor to acquire data indicative of the force exerted by each of the resected distal femur and the resected proximal tibia on the spacer block;
generating data indicative of a contact pressure on the spacer block based on the data acquired by the at least one sensor; and
using said data to determine a course of action associated with a total knee arthroplasty procedure.

8. The method according to claim 7, wherein said step of positioning a spacer block comprises positioning the spacer block adjacent the resected distal femur and the resected proximal tibia while the femur and tibia are fully extended.

9. The method according to claim 7, wherein said step of generating data comprises generating a two-dimensional image indicative of the contact pressure on the spacer block.

10. The method according to claim 7, further comprising removing the spacer block from the resected distal femur and resected proximal tibia when the contact pressure is within a predetermined range.

11. The method according to claim 10, further comprising positioning a femoral implant component adjacent to the resected distal femur and positioning a tibial implant component adjacent to the resected proximal tibia.

12. The method according to claim 7, further comprising resecting one of the resected distal femur and the resected proximal tibia in response to the contact pressure being higher than a predetermined amount.

13. The method according to claim 7, further comprising adding additional layers to one of a femoral implant component and a tibial implant component in response to the contact pressure being lower than a predetermined amount.

14. A force sensing system comprising:
a spacer block that is adapted to be positioned adjacent a resected distal femur and a resected proximal tibia, said spacer block being configured to receive one or more forces applied by at least one of said resected distal femur and said resected proximal tibia;
at least one sensor array that is associated with said spacer block and that is adapted to measure one or more forces exerted on said spacer block; and
a processing element configured to receive information from said sensor array and generate information indicative of the one or more forces exerted on said spacer block.

15. The force sensing system of claim 1, further comprising a display configured for displaying the information indicative of the one or more forces exerted on said spacer block.

16. The system according to claim 15, wherein said display is configured to display a two-dimensional image indicative of a contact pressure on said spacer block.

17. The system according to claim 15, wherein said display is configured to display an indication of an anisotropic contact pressure on said spacer block.

18. The system according to claim 14, wherein said at least one sensor array is integrated with said spacer block.

19. The system according to claim 14, wherein said at least one sensor block comprises a sensing portion and a gripping portion extending outwardly from said sensing portion, and wherein said at least one sensor array is associated with said sensing portion.

20. The system according to claim 14, where said at least one sensor array comprises a plurality of sensors each configured to measure one or more forces exerted on said spacer block applied by said resected distal femur and said resected proximal tibia.
